(19) United States
(12) Patent Application Publication
(10) Pub. No.: US 2023/0373943 A1
(43) Pub. Date: Nov. 23, 2023

(54) EXTRACTION OF CANNABINOIDS FROM WET BIOMASS

(71) Applicant: CHEMTOR, LP, Lockhart, TX (US)

(72) Inventor: William Lanier, West Jordan, UT (US)

(21) Appl. No.: 18/328,009

(22) Filed: Jun. 2, 2023

Related U.S. Application Data
(63) Continuation of application No. PCT/US2023/022502, filed on May 17, 2023.
(60) Provisional application No. 63/364,924, filed on May 18, 2022.

(51) Int. Cl. C07D 311/80; C07C 51/47; C07D 311/70; C07C 37/82; C07D 311/58; B01D 11/04; B01D 21/26

(52) U.S. Cl. CPC C07D 311/80; C07C 51/47; C07D 311/70; C07C 37/82; C07D 311/58; B01D 11/0492; B01D 21/262; C07C 2601/16

(57) ABSTRACT

A method of extracting cannabinoids includes providing wet biomass having cannabinoids and a water activity level of at least 0.55, contacting the wet biomass with an extractant to form an extractant mixture, removing solid biomass from the extractant mixture to form a liquid extract, and removing a cannabinoid-enriched oil layer from the liquid extract.

### CROSS REFERENCE TO RELATED APPLICATIONS

The present application is a continuation of PCT International Patent Application No. PCT/US2023/022502 filed May 17, 2023, and entitled “EXTRACTION OF CANNABINOIDS FROM WET BIOMASS,” which claims benefit of priority to U.S. Provisional Patent Application No. 63/364,924 filed May 18, 2022, entitled “EXTRACTION OF CANNABINOIDS FROM WET BIOMASS”, the disclosures of which are hereby incorporated by reference in their entirety.

### FIELD OF DISCLOSURE

The present disclosure is related to purification and extraction of cannabinoids. More particularly, this disclosure is related to methods and systems of extracting cannabinoids from wet biomass or plant material.

### BACKGROUND

Cannabinoids occur in the hemp plant, Cannabis sativa, primarily in the form of cannabinoid carboxylic acids (referred to herein as “cannabinoid acids”). The more abundant forms of cannabinoid acids include tetrahydrocannabinolic acid (THCA), cannabidiolic acid (CBDA), cannabigerolic acid (CBGA) and cannabichromic acid (CBCA). Other acid cannabinoids include, but are not limited to, tetrahydrocannabivaric acid (THCVA), cannabidivaric acid (CBDVA), cannabigerovaric acid (CBGVA) and cannabichromevaric acid (CBCVA). “Neutral cannabinoids” are derived by decarboxylation of their corresponding cannabinoid acids. The more abundant forms of neutral cannabinoids include tetrahydrocannabinol (THC), cannabidiol (CBD), cannabigerol (CBG) and cannabichromene (CBC). Other neutral cannabinoids include, but are not limited to, tetrahydrocannabivarin (THCV), cannabidivarin (CBDV), cannabigerovarin (CBGV), cannabichromevarin (CBCV) and cannabivarin (CBV). Cannabinoids may also include minor cannabinoids, including, for example, cannabielsoin (CBE) and cannabicyclol (CBL).

Cannabinoid oils can be extracted from plant material using a solvent. There are three primary types of solvent extraction: hydrocarbon, ethanol, and carbon dioxide. However, these solvent extractions tend to be most effective when the plant material has a lower amount of water. Therefore, most solvent extractions use dried plant material.

Fresh hemp plant contains approximately 80% water. To be considered dried, the plant material generally should have a water activity level of between 0.55-0.65 (ASTM D8196-18) or a water content of at most 5-15% depending on the application and regional laws. However, drying plant material is a time-consuming and expensive process.

There are several methods by which wet plant material is dried. One such method is air-drying, also known as hang-drying. In this method, the wet plant material is hung by a string or laid out in a cool, dark room. The temperature and humidity of the room must be controlled to prevent unwanted degradation of desirable compounds and the growth of mold. Air-drying generally takes between 7-10 days but can take up to a few weeks. Moreover, air-drying is expensive, labor-intensive, and requires large storage rooms.

Another method of drying wet plant material is oven-drying, in which wet plant material is heated in an oven, optionally under vacuum. This method is faster than air-drying and decreases the amount of labor and storage needed. However, the high temperatures can degrade or evaporate desired compounds, including cannabinoid oils, resulting in a less potent product.

Additionally, plant matter can be dried via freeze-drying. In this method, wet plant material is held at a low temperature to crystallize the water in the plant material. A vacuum is then applied to sublime the water out of the plant material. Compared to the aforementioned processes, freeze-drying better maintains the desired compounds, resulting in a more potent, and thus higher quality product. Additionally, this method can be completed in less than 24 hours. However, freeze-drying requires a lot of energy and equipment, making it very expensive.

Once plant material is dried, the dried plant material is often cured. During the curing process, the plant material is further dried and aged in a container, for example a sealed jar or can. Curing generally takes another two to four weeks.

There remains a need for an efficient process of extracting cannabinoid oils from plant material without undergoing the expensive and time-consuming steps of drying and/or curing wet plant material.

### BRIEF DESCRIPTION OF THE DRAWINGS

The following FIGURES illustrates embodiments of the subject matter disclosed herein. The claimed subject matter may be understood by reference to the following description taken in conjunction with the accompanying FIGURES, in which:

FIG. 1 is a diagram of an extraction process according to an embodiment of the present disclosure.

FIG. 2 is a table showing data from the Example.

### DETAILED DESCRIPTION

The following disclosure provides many different embodiments or examples. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

The present disclosure relates to the extraction of cannabinoids from wet biomass to create cannabinoid-enriched oil. Extraction of cannabinoids is generally performed using dried biomass. However, drying and curing fresh plant material is time-consuming and expensive. Therefore, by using wet biomass, the system and method of the present disclosure may reduce time and expense associated with the production of cannabinoid-enriched oil.

In the present disclosure, cannabinoids are extracted from the wet biomass using an extractant that is immiscible with water. The solid plant matter may then be removed, for example, via centrifugation or filtration. In some embodiments, the resulting liquid extractant mixture is flowed through a conduit contact reactor to separate an organic phase from an aqueous phase. The conduit contact reactor may be a fiber reactor as described in U.S. Pat. No. 11,198,107, which is hereby incorporated by reference in its entirety. In the fiber reactor, aqueous solution (e.g., water, caustic or acid) may be simultaneously introduced into the fiber reactor to improve separation and wash the organic phase. The organic phase that forms after passing through the fiber reactor is cannabinoid-enriched and may be the final product or may undergo further purification. The aqueous phase that forms after passing through the fiber reactor may be recycled, discarded, or further treated to, for example, isolate components thereof.

In some embodiments, the term “wet biomass” refers to cannabinoid-containing biomass or plant material with a water activity level (aw) of at least 0.55. For example, in some embodiments the wet biomass can have a minimum water activity of 0.60, 0.65, 0.70, 0.75, 0.80, 0.85, 0.90, 0.95, or 0.99. In other embodiments, the term “wet biomass” refers to cannabinoid-containing biomass or plant material with a water content of at least 5 wt %. For example, in some embodiments, the wet biomass can have a minimum water content of 5 wt %, 10 wt %, 20 wt %, 30 wt %, 40 wt %, 50 wt %, 60 wt %, 70 wt %, 80 wt %, or 90 wt %. In some embodiments, the cannabinoid-containing plant material includes fresh hemp plant, Cannabis sativa.

Wet biomass may include discarded plant material or water produced during a bubble hash process. In this process, trichomes, which form on the surface of the cannabis flower and contain most of the desirable compounds like cannabinoids and terpenes, are separated from the other plant material. The fresh cannabis plant is placed in ice water to separate the trichomes from the plant material. The leftover plant material is then filtered out from the water and trichome mixture. Usually, the leftover plant material is discarded. Next, the water and trichome are separated by allowing the trichome to settle to the bottom. The water is usually discarded as well. The discarded plant material and water may still contain cannabinoids. Thus, discarded plant material and water from the bubble hash process can be used as wet biomass in the extraction process described in this disclosure.

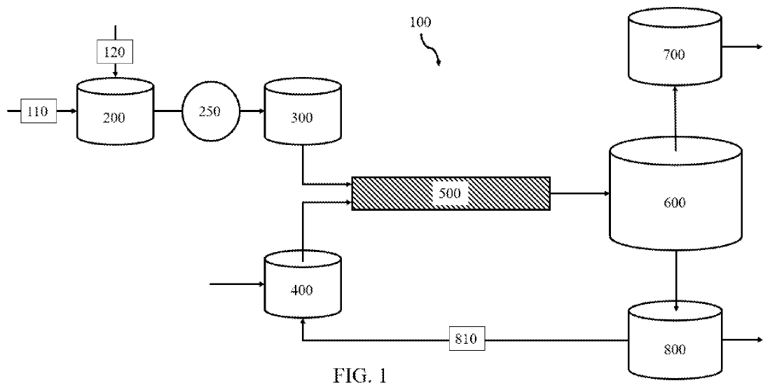

FIG. 1 illustrates a system 100 for extracting cannabinoids from wet biomass according to one embodiment of the present disclosure. In some embodiments, the system 100 may also extract other desirable compounds including terpenes, flavonoids, and carotenoids. An initial step may include adding wet biomass from a wet biomass supply 110 and an extractant from an extractant supply 120 to the extraction process 200. In the extraction process 200, the extractant extracts cannabinoids from the wet biomass. The extraction process 200 may include agitating the wet biomass and extractant in large mixing tanks. In some embodiments, extraction process 200 may include macerating the wet biomass and extractant. In other embodiments, the extractant may be flowed through the wet biomass. In other embodiments, the extractant and wet biomass may be processed through a centrifuge.

According to the present disclosure, the extractant is both immiscible with water and able to extract cannabinoids. In some embodiment, the extractant is a liquid hydrocarbon. For example, the extractant can include heptane, hexane, olive oil, methyl chloride, petroleum ether, methyl tert-butyl ether, ethyl acetate, chloroform, or combinations thereof. In some embodiments, the extractant is a mixture of two or more liquid hydrocarbons, such as those mentioned above. In some embodiments, the extractant is a hydrocarbon that is liquid under high pressure and/or at low temperature, such as butane or propane, and the extraction is performed at a pressure and temperature sufficient to maintain the extractant in liquid form.

The extraction process 200 may be run for any appropriate amount of time to allow sufficient contact between the extract and the wet biomass. Agitation, maceration, and the like may decrease the time needed for the extraction process 200. For example, the extraction process 200 may run for a period ranging from 5 minutes to 2 hours, about 15 minutes, about 30 minutes, about 1 hour, or 30 minutes to 1 hour. The extraction process 200 may be run at any appropriate temperature. For example, the temperature may be in the range of −80° C. to 70° C. and varies relative to the boiling point of the solvent being used. Elevated temperatures may decrease the extraction time, but the temperature should not be so high as to degrade (e.g., decarboxylate) components of the wet biomass. Moreover, the extraction process 200 may be run at any appropriate pressure. For example, the pressure may be in the range of 0 to 300 psi.

The wet biomass supply 110 may include whole or parts of leaves or buds or may be ground. In some embodiments, the wet biomass is ground before entering the extraction process 200. In another embodiment, the extractant may be mixed with the wet biomass, then the mixture may be ground together. In any embodiment, the wet biomass can be ground before or after entering extraction process 200.

The extraction mixture containing solid material is then moved from the extraction process 200 to a centrifuge 250 to remove the solid material. In other embodiments, the solid material can be separated by any appropriate process, including filtration using, for example, a sieve or filter. Alternatively, the extractant can simply be drained off the solid plant material, decanted or separated using a screw press or similar device. The liquid extractant mixture may be recovered from the centrifuge 250 and may be processed further as described below. The liquid extractant mixture may include at least some of the extractant added to the centrifuge and at least some of the water disposed in the wet biomass. In some embodiments, the liquid extractant mixture may include small pieces of solid material.

In some embodiments, the wet biomass supply 110 and the extractant supply 120 may be directly added to a centrifuge 250. The wet biomass may be disposed in a vessel that allows the extractant or water to flow into and out of the vessel without allowing the wet biomass to move out of the vessel. Thus, the extractant may be able to contact the wet biomass without the wet biomass escaping the vessel. The vessel may be a bag or other container and may be formed of mesh, cloth, sieve, filter bag and/or any other construct that allows the passage of solvent through the biomass while retaining material and particulates. In other embodiments, the wet biomass may not be disposed in a vessel.

The extractant may be allowed to contact the wet biomass for any suitable amount of time and may be agitated to improve contact therebetween. The wet biomass and extractant mixture may then be centrifuged to separate the liquid extractant mixture from the solid material. The solid material may be removed from the centrifuge 250. In some cases, a second batch of wet biomass may be input into the centrifuge 250 via the wet biomass supply 110. The liquid extractant mixture separated using the centrifuge 250 in the first batch may be left in the centrifuge 250 or may be recycled back into the centrifuge 250. Thus, cannabinoids may be extracted from multiple batches of wet biomass using the same liquid extractant mixture. In some embodiments, the same liquid extractant mixture may be used to extract cannabinoids from 1, 2, 3, 4, 5, 6, 7, 8, 9, or 10 batches of wet biomass.

Once the solid material is separated from the liquid extractant mixture after the last batch of wet biomass extraction in the centrifuge 250, the liquid extractant mixture is moved to holding tank 300. The liquid extraction mixture may naturally separate into an aqueous phase comprising the water content from the wet biomass and an organic phase comprising the extractant and cannabinoids from the wet biomass. In another embodiment, the liquid extraction mixture may be an emulsion of the organic phase and the aqueous phase. In yet another embodiment, the liquid extraction mixture may form three layers: an aqueous phase, an organic phase, and an emulsion phase between the aqueous and organic phases.

In some embodiments, the liquid extraction mixture is flowed through a fiber reactor 500 to separate the organic phase from the aqueous phase. While the liquid extraction mixture is flowed through the fiber reactor 500, an aqueous solution from holding tank 400 may be simultaneously flowed through the fiber reactor 500. In some embodiments, the aqueous solution may be brine. In other embodiments, the aqueous solution may be acidic. For example, the acidic aqueous solution may include one or more of hydrochloric acid, nitric acid, sulfuric acid, citric acid or any other food grade acid. In other embodiments, the solution may be basic. For example, a basic solution may include one or more of sodium hydroxide, potassium hydroxide, lithium hydroxide, calcium hydroxide, sodium oxide, magnesium oxide, copper oxide, ammonia, trimethyl amine, triethyl amine, pyridine, sodium acetate, sodium bicarbonate, sodium carbonate, sodium citrate or any other food grade base. When in the fiber reactor 500, the aqueous solution can remove compounds from the organic phase including chlorophyll, sugars, and phospholipids, heavy metals, and water-soluble pesticides. In some embodiments, an aqueous solution is not added to the fiber reactor 500. Instead, the water separating from the wet biomass can function as the aqueous solution, thus a separate aqueous solution need not be added in these embodiments. In other embodiments, an aqueous phase may be altogether absent in the fiber reactor 500.

The reaction product from the fiber reactor 500 is moved to a settling tank 600. In the settling tank 600, the organic phase and the aqueous phase form separate layers that can be separately removed from the settling tank 600.

In some embodiments, the cannabinoid-enriched oil (organic phase) is moved from the settling tank 600 to a holding tank 700. In some embodiments, a weight percentage of cannabinoids in the organic phase may range from 5% to 85% depending on the quality of the wet biomass supply 110 input and the use of the fiber reactor 500 to additionally increase the concentration of cannabinoids in the resulting oil by way performing a series of water washes to remove non-cannabinoid content, i.e., gums and phospholipids.

The cannabinoid-enriched oil may contain one or more of tetrahydrocannabinolic acid (THCA), cannabidiolic acid (CBDA), cannabigerolic acid (CBGA), cannabichromic acid (CBCA), tetrahydrocannabivaric acid (THCVA), cannabidivaric acid (CBDVA), cannabigerovaric acid (CBGVA), cannabichromevaric acid (CBCVA), tetrahydrocannabinol (THC), cannabidiol (CBD), cannabigerol (CBG) and cannabichromene (CBC), tetrahydrocannabivarin (THCV), cannabidivarin (CBDV), cannabigerovarin (CBGV), cannabichromevarin (CBCV), cannabivarin (CBV) or the inclusion of trace amount of minor cannabinoids, for example, cannabielsoin (CBE) and cannabicyclol (CBL). The cannabinoid-enriched oil may also contain one or more types of terpenes, flavonoids, and/or carotenoids.

The cannabinoid-enriched oil may undergo washes and separations to further purify the oil, optionally using a fiber reactor. These further purification processes may include one or more of de-gumming, de-waxing, winterization, decarboxylation, distillation, crystallization, decolorization/carbon scrubbing, vacuum purge, chromatography or further solvent separation.

In some embodiments, the aqueous phase is moved from the settling tank 600 to a holding tank 800. The aqueous phase, or wastewater, may contain undesirable compounds including one or more of chlorophyll, sugars, and phospholipids. However, the wastewater may also contain small amounts of desirable compounds including one or more of cannabinoids, terpenes, flavonoids, gums and carotenoids. Depending on the makeup of the wastewater, the wastewater may undergo further separation processes to isolate the desirable compounds. The wastewater may also be recycled to the aqueous solution holding tank 400 as shown by recycle stream 810. In some embodiments, all of the wastewater or a portion thereof may be recycled.

### Example

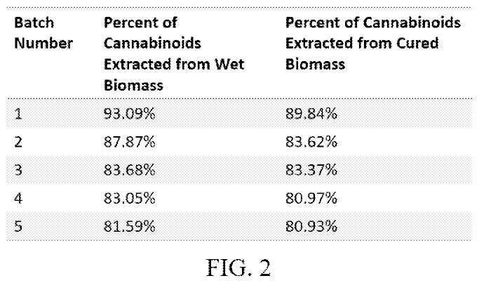

A liquid extractant mixture comprising heptane was used to extract cannabinoids from wet biomass and dried or cured biomass. Cannabinoids from 5 batches of each type of biomass were extracted using the same liquid extractant mixture; the extractant from the first batch was reused in each successive batch. For each batch, the percentage of cannabinoids that were extracted from the biomass was calculated. The percentage was calculated by measuring an amount of cannabinoids present in the batch of biomass and measuring an amount of cannabinoids in the solid material after extraction. These measurements were used to calculate the percentage of the total cannabinoids that were extracted by the liquid extractant mixture. FIG. 2 is a table including data collected from the Example. As shown, the percent of cannabinoids extracted by the liquid extractant mixture from the wet biomass is higher than the percent of cannabinoids extracted by the liquid extractant mixture from the cured biomass for every batch. Additionally, after 5 batches, the liquid extractant mixture extracts a high percentage of the cannabinoids in the wet biomass. Therefore, the extraction process described herein may be used to generate a high-quality cannabinoid oil with a higher percentage of cannabinoids.

The liquid extractant mixture or cannabinoid oil end product may be a golden color and the color may be light, which are characteristics that may be associated with a high-quality end product. In some cases, the extraction process may extract a higher percentage of cannabinoids from wet biomass, thus avoiding many of the drawbacks of curing biomass described herein. Moreover, reusing the same liquid extractant mixture may decrease the cost and waste associated with the extraction process while generating a high-quality cannabinoid oil.

Although various embodiments have been shown and described, the disclosure is not limited to such embodiments and will be understood to include all modifications and variations as would be apparent to one of ordinary skill in the art. Therefore, it should be understood that the disclosure is not intended to be limited to the particular forms disclosed; rather, the intention is to cover all modifications, equivalents, and alternatives falling within the spirit and scope of the disclosure as defined by the appended claims.

What is claimed is:

1. A method of extracting cannabinoids, comprising:
providing wet biomass comprising cannabinoids and having a water activity level of at least 0.55;
contacting the wet biomass with an extractant to form an extractant mixture, wherein the extractant is immiscible with water and capable of extracting cannabinoids;
removing solid biomass from the extractant mixture to form a liquid extract; and
separating an organic phase comprising the extractant and cannabinoids from the liquid extract.

2. The method of claim 1, wherein separating comprises:
flowing the liquid extract through a conduit contact reactor to form a reaction product comprising an aqueous phase comprising water from the wet biomass and the organic phase, the conduit contact reactor comprising a hollow conduit having a plurality of fibers disposed therein;
collecting the reaction product in a separator; and
separately removing the aqueous phase from the organic phase.

3. The method of claim 2, further comprising the step of:
flowing an aqueous solution through the conduit contact reactor while simultaneously flowing the liquid extract through the conduit contact reactor.

4. The method of claim 3, wherein the aqueous solution is a brine.

5. The method of claim 4, wherein the brine contains an acid or a base.

6. The method of claim 1, wherein the organic phase comprises one or more of tetrahydrocannabinolic acid (THCA), cannabidiolic acid (CBDA), cannabigerolic acid (CBGA), cannabichromic acid (CBCA), tetrahydrocannabivaric acid (THCVA), cannabidivaric acid (CBDVA), cannabigerovaric acid (CBGVA), cannabichromevaric acid (CBCVA), tetrahydrocannabinol (THC), cannabidiol (CBD), cannabigerol (CBG), cannabichromene (CBC), tetrahydrocannabivarin (THCV), cannabidivarin (CBDV), cannabigerovarin (CBGV), cannabichromevarin (CBCV) or cannabivarin (CBV).

7. The method of claim 1, wherein the extractant is heptane, hexane, olive oil, methyl chloride, petroleum ether, or methyl tert-butyl ether, chloroform, ethyl acetate, or combinations thereof.

8. The method of claim 1, wherein the extractant is heptane.

9. The method of claim 1, wherein the wet biomass has a water content of at least 5 wt %.

10. The method of claim 1, wherein the solid biomass is removed via centrifugation.

11. A system for extracting cannabinoids from wet biomass, comprising:
a wet biomass supply comprising wet biomass comprising cannabinoids and having a water activity level of at least 0.55;
an extractant supply comprising an extractant that is immiscible with water and capable of extracting cannabinoids; and
an extraction process configured to receive the wet biomass from the wet biomass supply and the extractant from the extractant supply and form an extraction mixture comprising solid biomass and a liquid extractant;
wherein the liquid extractant comprises at least a portion of a water content from the wet biomass, at least a portion of the cannabinoids from the wet biomass, and the extractant; and
wherein the solid biomass comprises the wet biomass less the at least a portion of the water content and the at least a portion of the cannabinoids.

12. The system of claim 11, further comprising:
a conduit contact reactor comprising a plurality of fibers disposed therein and configured to receive the liquid extractant;
a settling tank configured to receive a reaction product from the conduit contact reactor;
wherein the reaction product comprises an organic layer and an aqueous layer, the organic layer comprising the extractant and at least a portion of the cannabinoids from the wet biomass.

13. The system of claim 11, wherein the extraction process comprises a centrifuge.

14. The system of claim 12, wherein the conduit contact reactor is configured to receive an aqueous solution and the extractant mixture simultaneously.

15. A method of extracting cannabinoids, comprising:
providing a wet biomass comprising cannabinoids and having a water content of at least 10 wt %;
removing solid biomass from the wet biomass to form a liquid extract; and
separating an organic phase comprising cannabinoids from the liquid extract by flowing the liquid extract through a conduit contact reactor comprising a conduit having a plurality of fibers disposed therein.

16. The method of claim 15, wherein providing the wet biomass comprises contacting cannabis plant matter with ice water to remove trichromes therefrom and separating the water and trichrome mixture from the wet biomass.

17. The method of claim 15, wherein an aqueous solution is flowed through the conduit contact reactor while the liquid extract is flowed through the conduit contact reactor.

18. The method of claim 15, wherein the solid biomass is removed via centrifugation.

19. The method of claim 17, wherein the aqueous solution is a brine.

20. The method of claim 19, wherein the brine comprises an acid or a base.
